(19) United States
(12) Patent Application Publication
(10) Pub. No.: US 2008/0131503 A1
(43) Pub. Date: Jun. 5, 2008

(54) Stable Pharmaceutical Composition Comprising a Fixed Dose Combination of Fenofibrate and an Hmg-Coa Reductase Inhibitor

(76) Inventors: Per Holm, Vanlose (DK); Tomas Norling, Lyngby (DK)

Correspondence Address: DARBY & DARBY P.C., P.O. BOX 770, Church Street Station, New York, NY, 10008-0770, US

(21) Appl. No.: 10/582,410

(22) PCT Filed: Feb. 10, 2006
(86) PCT No.: PCT/DK06/50004

§ 371 Date: Sep. 19, 2006

(30) Foreign Application Priority Data
Feb. 10, 2005 (DK) PA 2005 00200
Apr. 20, 2005 (DK) PA 2005 00576

(51) Int. Cl. A61K 31/216; A61K 9/28; A61K 9/16; A61K 31/40; A61P 43/00; A61K 31/366; A61K 9/50

(52) U.S. Cl. 424/463; 424/474; 424/490; 514/513; 514/460; 514/423

(57) ABSTRACT

A pharmaceutical composition for oral administration comprising a fixed dose combination of a first solid pharmaceutical composition containing fenofibrate as the active substance and second solid pharmaceutical composition containing an HMG-CoA reductase inhibitor such as a statin as the active substance, wherein the first and the second pharmaceutical compositions are present in separate entities in a single solid dosage form. For example a multilayer tablet, a two-layer tablet, or capsules or sachets containing the active ingredients in separate granulates or beads, either granulate or bead optionally being coated with a protective coating or an entero-coating.

The invention relates to a stable pharmaceutical composition comprising at least two active pharmaceutical ingredients, namely fenofibrate as a first ingredient and an HMG CoA reductase inhibitor or a derivative thereof as a second ingredient. More specifically, the invention relates to a single solid dosage form for oral administration comprising a solid fenofibrate composition and a solid HMG-CoA reductase inhibitor composition, preferably a statin composition, the active substances being present in separate entities.

### BACKGROUND OF THE INVENTION

Clinical guidelines indicate that not only fibrate therapy but also a combination therapy with e.g. fenofibrate and a statin should be the most effective means of cholesterol and lipid management. In fact, treatment with fenofibrate is often prescribed together with a statin as clinicians seem to prefer the use of e.g. fenofibrate due to its triglyceride-lowering and HDL-C increasing effects while a statin is used for its positive effects on lowering LDL-C and raising HDL-C. However, at present, such a combination therapy can only be achieved by the use of two separate products, i.e. the patient needs to take e.g. one fenofibrate tablet or capsule together with another tablet or capsule containing a statin.

Fenofibrate is chemically named 2-[4-(4-chlorobenzoyl]-2-methyl-propanoic acid, 1-methylethyl ester. Fenofibric acid produces reductions in total cholesterol (total-C), LDL-C, apo-lipoprotein B, total triglycerides, and triglyceride rich lipoprotein (VLDL) in treated patients. In addition, treatment with fenofibrate results in increases in high density lipoprotein (HDL) and apo-lipoprotein apoAl and apo All. Fenofibrate acts as a potent lipid regulating agent offering unique and clinical advantages over existing products in the fibrate family of drug substances. Fenofibrate produces substantial reduction in plasma triglyceride levels in hypertriglyceridemic patients and in plasma cholesterol and LDL-C in hypercholesterolemic and mixed dyslipidemic patients.

Statins are HMG CoA reductase inhibitors. Useful statins include lovastatin, fluvastatin, rosuvastatin, pravastatin, atorvastatin and simvastatin.

WO 2005/034908 discloses a combination of fenofibrate and a statin in a single dosage form.

However, certain statins are known to be susceptible to degradation and/or oxidation when subjected to unfavorable physical and/or chemical conditions. Also, an optimized combination drug product may call for different release profiles of each of the active substances.

Accordingly, there is an unmet need for providing a single dosage form comprising a combination of fenofibrate and a statin, in which all active pharmaceutical substances remain stable and wherein the active substances are provided in a formulation providing maximum bioavailability and/or maximum therapeutic or pharmacological response.

### SUMMARY OF THE INVENTION

The inventors have found that a fixed dose drug combination product comprising fenofibrate and an HMG-CoA reductase inhibitor can advantageously be prepared as a single solid dosage form in such a manner that the two active drug substances are present in separate entities. Thus, the active substances are effectively prevented from any drug-drug interaction; the active substances may independently of each other be provided in different release forms, i.e. in the form of immediate release, delayed release or controlled release compositions; and the stability of the combination drug product can be maximized due to the possibility of optimizing the formulations of each of the active substances with respect to physical and/or chemical conditions.

Accordingly, in a first aspect the invention relates to a pharmaceutical composition for oral administration comprising a first solid pharmaceutical composition containing fenofibrate as the active substance and second solid pharmaceutical composition containing an HMG-CoA reductase inhibitor as the active substance, wherein the first and the second pharmaceutical composition are present in separate entities in a single solid dosage form.

In a second aspect, the invention relates to a pharmaceutical composition for the treatment of a subject suffering from atherosclerosis, hyperlipidemia, and/or hypercholesterolemia.

In a third aspect, the invention relates to a method of manufacturing the pharmaceutical composition of the invention in a solid oral dosage form, for example a multilayer tablet.

In a further aspect, the invention relates to a single solid dosage form comprising the pharmaceutical composition of the invention.

### DETAILED DESCRIPTION OF THE INVENTION

### Definitions

As used herein, the term “active substance”, “active pharmaceutical substance”, “active ingredient” or “active pharmaceutical ingredient” means any component that is intended to furnish pharmacological activity or other direct effect in the diagnosis, cure, mitigation, treatment, or prevention of disease, or to affect the structure or any function of the body of man or other animals. The term includes those components that may undergo chemical change in the manufacture of the drug product and are present in the drug product in a modified form intended to furnish the specified activity or effect.

As used herein, the term “vehicle” means any solvent or carrier in a pharmaceutical product that has no pharmacological role. For example, water is the vehicle for xylocaine and propylene glycol is the vehicle for many antibiotics.

In the present context, the term “solid dispersion” denotes a drug or active ingredient or substance dispersed on a particulate level in an inert vehicle, carrier, diluent or matrix in the solid state, i.e. usually a fine particulate dispersion.

In the present context, the term “solid solution” denotes a drug or active ingredient or substance dissolved on a molecular level in an inert vehicle, carrier, diluent or matrix in the solid state.

As used herein, the term “analog” means a chemical compound that is structurally similar to another.

The term “drug” means a compound intended for use in diagnosis, cure, mitigation, treatment, or prevention of disease in man or other animals.

In this context, the term “dosage form” means the form in which the drug is delivered to the patient. This could be parenteral, topical, tablet, oral (liquid or dissolved powder), suppository, inhalation, transdermal, etc.

As used herein, the term “bioavailability” denotes the degree means to which a drug or other substance becomes available to the target tissue after administration. In the present context, the term “suitable bioavailability” is intended to mean that administration of a composition according to the invention will result in a bioavailability that is improved compared to the bioavailability obtained after administration of the active substance(s) in a plain tablet; or the bioavailability is at least the same or improved compared to the bioavailability obtained after administration of a commercially available product containing the same active substance(s) in the same amounts. In particular it is desired to obtain quicker and larger and/or more complete uptake of the active compound, and thereby provide for a reduction of the administered dosages or for a reduction in the number of daily administrations. Further, pharmaceutical compositions of the invention may also reduce or negate the need for food to be takes simultaneously with the dosage form (in particular relevant for one or the active substances contained in a composition of the invention, namely fenofibrate) thereby allowing patients more freedom on when the drug is taken.

In this context, the term “medicine” means a compound used to treat disease, injury or pain. Medicine is designated “prophylactic,” i.e. the art of preserving health, and “therapeutic”, i.e. the art of restoring health.

In the present context, the terms “controlled release” and “modified release” are intended to be equivalent terms covering any type of release of fenofibrate or statin from a composition of the invention that is appropriate to obtain a specific therapeutic or prophylactic response after administration to a subject. A person skilled in the art knows how controlled release/modified release differs from the release of plain tablets or capsules. The terms “release in a controlled manner” or “release in a modified manner” have the same meaning as stated above. The terms include slow release (that results in a lower Cₘₐₓ and later tₘₐₓ, but the half-life remains unchanged), extended release (that results in a lower Cₘₐₓ, later tₘₐₓ, but apparent half-life is longer); delayed release (that result in an unchanged Cₘₐₓ, but lag time and, accordingly, tₘₐₓ is delayed, and the half-life remains unchanged) as well as pulsatile release, burst release, sustained release, prolonged release, chrono-optimized release, fast release (to obtain an enhanced onset of action) etc. Included in the terms is also e.g. utilization of specific conditions within the body e.g., different enzymes or pH changes in order to control the release of the drug substance.

In this context, the term “erosion” or “eroding” means a gradual breakdown of the surface of a material or structure, for example of a tablet or the coating of a tablet.

In a first aspect, the invention relates to pharmaceutical composition for oral administration comprising a first solid pharmaceutical composition containing fenofibrate as the active substance and second solid pharmaceutical composition containing an HMG-CoA reductase inhibitor as the active substance, wherein the first and the second pharmaceutical composition are present in separate entities in a single solid dosage form. The HMG-CoA reductase inhibitor is a statin selected from the group consisting of atorvastatin, lovastatin, pravastatin, simvastatin, rosuvastatin, fluvastatin and pitavastatin.

In a preferred embodiment, the first solid pharmaceutical composition and/or the second solid pharmaceutical composition is in the form of granulate, granules, grains, beads or pellets, which are mixed and filled into capsules or sachets or are compressed to tablets by conventional methods. The granulate, granules, grains, beads or pellets containing the statin are optionally entero-coated or coated with a protective coating.

In another preferred embodiment, there is provided a tablet in which the first and second pharmaceutical compositions are present in at least two separate layers, i.e. a bi-layer or multilayer tablet. The layers comprising the first and second pharmaceutical compositions may be separated by an intermediate, inactive layer, for example a layer comprising one or more disintegrants.

In another aspect, the invention provides a method for preparing a single solid dosage form comprising a first solid pharmaceutical composition containing fenofibrate as the active substance and second solid pharmaceutical composition containing an HMG-CoA reductase inhibitor as the active substance, the first and the second pharmaceutical composition being present in separate entities, which method comprising the steps of:

i) preparing the first solid pharmaceutical composition,
ii) preparing the second solid pharmaceutical composition, and
iii) compressing the first and second compositions into a multilayer tablet, the first and second compositions being present in separate layers.

### The Active Drug Substances

A first drug or active substance of the dosage forms and pharmaceutical compositions of this invention is fenofibrate as described above or an analog thereof. However, it should be understood that this invention includes dosage forms and compositions comprising a mixture of two, three or even four different fibrates and/or fibric acids. Examples of other useful fibrates are bezafibrate, ciprofibrate, clinofibrate, clofibrate, etofylline, clofibrate, fenofibrate, gemfibrozil, pirifibrate, simfibrate and tocofibrate; particularly useful are gemfibrozil, fenofibrate, bezafibrate, clofibrate, ciprofibrate and active metabolites and analogues thereof including any relevant fibric acid such as fenofibric acid.

A second drug or active substance of the dosage forms and pharmaceutical compositions of this invention is an HMG-CoA reductase inhibitor or a derivative thereof, for example a statin selected from the group consisting of atorvastatin, fluvastatin, pravastatin, lovastatin, rosuvastatin and simvastatin and pharmaceutically acceptable salts thereof. For example, simvastatin is butanoic acid, 2,2-dimethyl-, 1,2,3,7,8,8a-hexahydro-3,7-dimethyl-8-[2-(tetrahydro-4-hydroxy-6-oxo-2H-pyran-2-yl)-ethyl]-1-naphthalenyl ester, [1S-[1(alpha),3(alpha),7(beta),8(beta)(2S*,4S*),-8a(beta)]]. The empirical formula of simvastatin is C₂₅H₃₈O₅ and its molecular weight is 418.57.

Simvastatin is a white to off-white, nonhygroscopic, crystalline powder that is practically insoluble in water, and freely soluble in chloroform, methanol and ethanol. Elevated plasma levels of total cholesterol (total-C), LDL-C, and apolipoprotein B (Apo B) promote human atherosclerosis and are risk factors for developing cardiovascular disease, while increased levels of high-density lipoprotein cholesterol (HDL-C) and its transport complex, Apo A-1, are associated with decreased cardiovascular risk. High plasma triglycerides (TG) and cholesterol-enriched TG-rich lipoproteins, including very-low-density lipoproteins (VLDL), intermediate-density lipoproteins (IDL), and remnants, can also promote atherosclerosis. Elevated plasma TG is frequently found in a triad with low HDL-C and small LDL particles, as well as in association with non-lipid metabolic risk factors for CHD. As such, total plasma TG has not consistently been shown to be an independent risk factor for CHD. Furthermore, the independent effect of raising HDL-C or lowering TG on the risk of coronary and cardiovascular morbidity and mortality has not been determined. Simvastatin has been shown to reduce both normal and elevated LDL-C concentrations. LDL is formed from very-low-density lipoprotein (VLDL) and is catabolized predominantly by the high-affinity LDL receptor. Simvastatin undergoes extensive first-pass extraction in the liver, its primary site of action, with subsequent excretion of drug equivalents in the bile. As a consequence of extensive hepatic extraction of simvastatin (estimated to be >60% in man), the availability of drug to the general circulation is low.

Atorvastatin is a synthetic lipid-lowering agent. Atorvastatin is an inhibitor of 3-hydroxy-3-methylglutaryl-coenzyme A (HMG-CoA) reductase. This enzyme catalyzes the conversion of HMG-CoA to mevalonate, an early and rate-limiting step in cholesterol biosynthesis. Atorvastatin is useful for example as the calcium salt, i.e. [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate. The molecular weight of atorvastatin calcium is 1209.42. Atorvastatin calcium is a white to off-white crystalline powder that is insoluble in aqueous solutions of pH 4 and below. Atorvastatin calcium is very slightly soluble in distilled water, pH 7.4 phosphate buffer, and acetonitrile, slightly soluble in ethanol, and freely soluble in methanol. However, atorvastatin is also useful as the magnesium salt. The atorvastatin salts may be either in crystalline form or in amorphous form or in a mixture of crystalline and amorphous form.

Atorvastatin is rapidly absorbed after oral administration; maximum plasma concentrations occur within 1 to 2 hours. Extent of absorption increases in proportion to atorvastatin dose. The absolute bioavailability of atorvastatin (parent drug) is approximately 14% and the systemic availability of HMG-CoA reductase inhibitory activity is approximately 30%. The low systemic availability is attributed to presystemic clearance in gastrointestinal mucosa and/or hepatic first-pass metabolism. Although food decreases the rate and extent of drug absorption by approximately 25% and 9%, respectively, as assessed by Cmax and AUC, LDL-C reduction is said to be similar whether atorvastatin is given with or without food. Plasma atorvastatin concentrations are lower (approximately 30% for Cmax and AUC) following evening drug administration compared with morning. However, LDL-C reduction is said to be the same regardless of the time of day of drug administration

### Pharmaceutically Acceptable Excipients and Additives

In the present context the term “pharmaceutically acceptable excipient” is intended to denote any material, which is inert in the sense that it substantially does not have any therapeutic and/or prophylactic effect per se. Such excipients may be added with the purpose of making it possible to obtain a pharmaceutical, cosmetic and/or foodstuff composition, which have acceptable technical properties. A particulate material or a solid dosage form according to the invention may contain one or more pharmaceutically acceptable excipients.

Examples of suitable excipients for use in a composition or solid dosage form according to the invention include fillers, diluents, disintegrants, binders, stabilizers, lubricants etc. or mixtures thereof. As the composition or solid dosage form according to the invention may be used for different purposes, the choice of excipients is normally made taken such different uses into considerations. Other pharmaceutically acceptable excipients for suitable use are e.g. acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

It is well-known that statins are pharmacologically active in the hydroxy acid form, whereas the corresponding lactone form may be considered a prodrug which may convert to the active hydroxy acid in vivo.

The active ingredient atorvastatin is included in the pharmaceutical composition as a salt of the pharmacologically active hydroxy acid form, preferably the hemi-calcium salt or the magnesium salt, in crystalline or amorphous form. In a preferred embodiment of the invention, atorvastatin is used in the crystalline magnesium salt form.

The atorvastatin hydroxy acid form—lactone form equilibrium and interconversion kinetics is pH highly dependent. The acid-catalyzed reaction is reversible, whereas the base-catalyzed reaction is practically irreversible: At pH>6, the equilibrium reaction is not detectable and greatly favors the hydroxy acid form (Kearney et al., Pharmaceutical Research, 1993, vol. 10, no. 10, p. 1461-65).

Accordingly, it is advisable to establish a near-neutral or basic microenvironment for atorvastatin in the pharmaceutical composition in order to stabilize the equilibrium, i.e. avoid presence of the inactive lactone form, for example an microenvironment having a pH above about 5 or even a pH above about 6.

It is known to incorporate a pharmaceutically acceptable inorganic alkalizing compound into a pharmaceutical composition comprising atorvastatin as a stabilizer. Such inorganic alkalizing compounds are typically conventional basic salts of metals or alkaline earth metals, for example calcium salts (calcium carbonate, calcium hydroxide, di calcium phosphate, tri calcium phosphate), magnesium salts (magnesium carbonate, magnesium hydroxide, magnesium silicate, magnesium aluminate, and aluminum magnesium hydroxide), lithium salts (lithium hydroxide), potassium salts (potassium hydroxide) and sodium salts (sodium bicarbonate, sodium borate, sodium carbonate, sodium hydroxide). Conventionally, the basic inorganic salts of calcium, lithium or magnesium are utilized in a weight ratio ranging between about 0.1 to 1 and about 50 to 1 of salt compound to atorvastatin (i.e. the active ingredient). Typically, calcium carbonate is used in an amount of at least 5% w/w of the pharmaceutical composition and even up to as much as about 70% w/w, typically in a w/w ratio atorvastatin-calcium carbonate of between 1:1 and 4:1. Without being bound to this theory, it is contemplated that it is necessary to use a high amount of calcium carbonate due to the low water solubility of calcium carbonate, below 0.1 mg/mL at neutral pH.

Other useful pharmaceutically acceptable inorganic compounds are for example talc and bentonite.

However, a basic or near-neutral microenvironment for atorvastatin may also be established by incorporating one or more pharmaceutically acceptable organic alkalizing compounds into the pharmaceutical composition. Useful organic compounds include amines, amides and ammonium compounds. Specific examples are ammonia, ammonium lactate, ammonium bicarbonate, ammonium hydroxide, ammonium phosphate dibasic, mono ethanolamine, di ethanolamine, tri ethanolamine, tri hydroxymethylaminomethane, ethylenediamine, N-methyl glucamide, 6N-methyl glucamine, meglucamine and L-lysine. A preferred compound is trometamol (IUPAC name: 2-amino-2-(hydroxymethyl)-1,3-propanediol; also known as tris buffer, tham, tromethamine, trisaminol or trisamine). Trometamol is useful in an amount of below 10% w/w of the pharmaceutical composition, preferably below 5% w/w. Typically, trometamol is used in the pharmaceutical composition comprising atorvastatin in an amount of at the most about 1% w/w of the composition. In a preferred embodiment of the invention, trometamol is used in an amount of below 1% w/w of the invention, preferably below 0.8% W/w, more preferably below 0.7% w/w, even more preferably below about 0.6% w/w, such as about 0.5% w/w, of the composition.

Examples of suitable fillers, diluents and/or binders include lactose (e.g. spray-dried lactose, a-lactose, b-lactose, Tabletose®, various grades of Pharmatose®, Microtose® or Fast-Floc®), microcrystalline cellulose (various grades of Avicel®, Elcema®, Vivacel®, Ming Tai® or Solka-Floc®), hydroxypropylcellulose, L-hydroxypropylcellulose (low substituted), hydroxypropyl methylcellulose (HPMC) (e.g., Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol (e.g. Pearlitol 50C), dextrins, maltodextrins, starches or modified starches (including potato starch, maize starch and rice starch), calcium phosphate (e.g., basic calcium phosphate, calcium hydrogen phosphate, dicalcium phosphate hydrate), calcium sulfate, calcium carbonate, sodium alginate, collagen etc.

Specific examples of diluents are e.g., calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants are e.g. alginic acid or alginates, microcrystalline cellulose, hydroxypropyl cellulose and other cellulose derivatives, croscarmellose sodium (Ac-di-sol), crospovidone, polacrillin potassium, sodium starch glycolate, starch, pregelatinized starch, carboxymethyl starch (e.g. Primogel® and Explotab®) etc.

Specific examples of binders are e.g., acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Glidants and lubricants may also be included in the first or, preferably, the second (statin-containing) composition. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients which may be included in a composition or solid dosage form of the invention are e.g., flavoring agents, coloring agents, taste-masking agents, pH-adjusting agents, buffering agents, preservatives, stabilizing agents, anti-oxidants, wetting agents, humidity-adjusting agents, surface-active agents (e.g. Polysorbate 80/Tween 80), suspending agents, absorption enhancing agents, agents for modified release etc.

Other additives in a composition or a solid dosage form according to the invention may be antioxidants like e.g. ascorbic acid, ascorbyl palmitate, butylated hydroxyanisole, butylated hydroxytoluene, citric acid, hypophosphorous acid, monothioglycerol, potassium metabisulfite, propyl gallate, sodium formaldehyde sulfoxylate, sodium metabisulfite, sodium thiosulfate, sulfur dioxide, tocopherol, tocopherol acetate, tocopherol hemisuccinate, TPGS or other tocopherol derivatives, etc. The concentration of antioxidants in the carrier composition is normally from about 0.1% w/w to about 5% w/w.

A composition or solid dosage form according to the invention may also include one or more surfactants or substances having surface-active properties. It is contemplated that such substances are involved in the wetting of the slightly soluble active substance and thus, contributes to improved solubility characteristics of the active substance. Suitable surfactants for use in a composition or a solid dosage form according to the invention are surfactants such as, e.g., hydrophobic and/or hydrophilic surfactants as those disclosed in WO 00/50007 in the name of Lipocine, Inc.

Specific examples of suitable surfactants are polyethoxylated fatty acids such as, e.g., fatty acid mono- or diesters of polyethylene glycol or mixtures thereof such as, e.g., mono- or diesters of polyethylene glycol with lauric acid, oleic acid, stearic acid, myristic acid, ricinoleic acid, and the polyethylene glycol may be selected from PEG 4, PEG 5, PEG 6, PEG 7, PEG 8, PEG 9, PEG 10, PEG 12, PEG 15, PEG 20, PEG 25, PEG 30, PEG 32, PEG 40, PEG 45, PEG 50, PEG 55, PEG 100, PEG 200, PEG 400, PEG 600, PEG 800, PEG 1000, PEG 2000, PEG 3000, PEG 4000, PEG 5000, PEG 6000, PEG 7000, PEG 8000, PEG 9000, PEG 1000, PEG 10,000, PEG 15,000, PEG 20,000, PEG 35,000, polyethylene glycol glycerol fatty acid esters, i.e. esters like the above-mentioned but in the form of glyceryl esters of the individual fatty acids; glycerol, propylene glycol, ethylene glycol, PEG or sorbitol esters with e.g., vegetable oils like e.g., hydrogenated castor oil, almond oil, palm kernel oil, castor oil, apricot kernel oil, olive oil, peanut oil, hydrogenated palm kernel oil and the like, polyglycerized fatty acids like e.g., polyglycerol stearate, polyglycerol oleate, polyglycerol ricinoleate, polyglycerol linoleate, propylene glycol fatty acid esters such as, e.g., propylene glycol monolaurate, propylene glycol ricinoleate and the like, mono- and diglycerides like e.g. glyceryl monooleate, glyceryl dioleae, glyceryl mono- and/or dioleate, glyceryl caprylate, glyceryl caprate etc.; sterol and sterol derivatives; polyethylene glycol sorbitan fatty acid esters (PEG-sorbitan fatty acid esters) such as esters of PEG with the various molecular weights indicated above, and the various Tween® series (from ICI America, Inc.); polyethylene glycol alkyl ethers such as, e.g., PEG oleyl ether and PEG lauryl ether; sugar esters like e.g. sucrose monopalmitate and sucrose monolaurate; polyethylene glycol alkyl phenols like e.g. the Triton® X or N series (Union Carbide Chemicals & Plastics Technology Corporation); polyoxyethylene-polyoxypropylene block copolymers such as, e.g., the Pluronic® series from BASF Aktiengesellschaft, the Synperonic® series from ICI America, Inc., Emkalyx, Lutrol® from BASF Aktiengesellschaft, Supronic etc. The generic term for these polymers is “poloxamers” and relevant examples in the present context are Poloxamer 105, 108, 122, 123, 124, 181, 182, 183, 184, 185, 188, 212, 215, 217, 231, 234, 235, 237, 238, 282, 284, 288, 331, 333, 334, 335, 338, 401, 402, 403 and 407; sorbitan fatty acid esters like the Span® series (from ICI) or Arlacel® series (from ICI) such as, e.g., sorbitan monolaurate, sorbitan monopalmitate, sorbitan monooleate, sorbitan monostearate etc.; lower alcohol fatty acid esters like e.g., oleate, isopropyl myristate, isopropyl palmitate etc.; ionic surfactants including cationic, anionic and zwitterionic surfactants such as, e.g., fatty acid salts, bile salts, phospholipids, phosphoric acid esters, carboxylates, sulfates and sulfonates etc.

When a surfactant or a mixture of surfactants is present in a composition or a solid dosage form of the invention, the concentration of the surfactant(s) is normally in a range of from about 0.1-80% w/w such as, e.g., from about 0.1 to about 20% w/w, from about 0.1 to about 15% w/w, from about 0.5 to about 10% w/w, or alternatively, from about 0.10 to about 80% w/w such as, e.g. from about 10 to about 70% w/w, from about 20 to about 60% w/w or from about 30 to about 50% w/w.

In a specific aspect of the invention, the at least one of the one or more pharmaceutically acceptable excipient is selected from the group consisting of silica acid or a derivative or salt thereof including silicates, silicon dioxide and polymers thereof; magnesium aluminosilicate and/or magnesium aluminometasilicate, bentonite, kaolin, magnesium trisilicate, montmorillonite and/or saponite.

### Solid Dosage Form Design

### Method of Manufacture

The first solid composition of the invention may be prepared by any method suitable for incorporation of poorly water-soluble active substances. The pharmaceutical compositions may be prepared by any convenient method such as, e.g. granulation, mixing, spray drying etc. A particularly useful method is the method disclosed in Applicants' co-pending international application published as WO 03/004001, which describes a process for preparation of particulate material by a controlled agglomeration method, i.e. a method, which enables a controlled growth in particle size. The method involves spraying a first composition comprising the active substance and a vehicle in liquid form onto a solid carrier. Normally, the vehicle has a melting point of at least 5° C., but the melting point must indeed be below the melting point of the active substance. In the present invention, the melting point of the vehicle and should not exceed 250° C.

It is within the skills of the average practitioner to select a suitable vehicle being pharmaceutical acceptable, capable of dispersing or fully or at least partly dissolving the active substance and having a melting point in the desired range using general knowledge and routine experimentation. Suitable candidate for carriers are described in WO 03/004001, which is herein incorporated by reference.

In the present context, suitable vehicles are e.g., those mentioned as vehicles or as oily materials as well as those disclosed in WO 03/004001. An advantage of using the controlled agglomeration method described in WO 03/004001 is that it is possible to apply a relatively large amount of a liquid system to a particulate material without having an undesirable growth in particle size. Accordingly, in one embodiment of the invention, the particulate material of a pharmaceutical composition has a geometric weight mean diameter dgw of 210 mm such as, e.g. 220 mm, from about 20 to about 2000, from about 30 to about 2000, from about 50 to about 2000, from about 60 to about 2000, from about 75 to about 2000 such as, e.g. from about 100 to about 1500 mm, from about 100 to about 1000 mm or from about 100 to about 700 mm, or at the most about 400 mm or at the most 300 mm such as, e.g., from about 50 to about 400 mm such as, e.g., from about 50 to about 350 mm, from about 50 to about 300 mm, from about 50 to about 250 mm or from about 100 to about 300 mm.

The first compositions of the invention are preferably formed by spray drying techniques, controlled agglomeration, freeze-drying or coating on carrier particles or any other solvent removal process. The dried product contains the active substance present preferably in dissolved form either fully dissolved as a solid solution or partly dissolved as a solid dispersion including a molecular dispersion and a solid solution.

The first composition of the invention may preferably be manufactured using a method comprising the steps of:

i) bringing the vehicle in liquid form, i.e. melting the vehicle if solid at room temperature,
ii) maintaining the liquid vehicle at a temperature below the melting point of the fibrate,
iii) dissolving the desired amount of fibrate in the vehicle,
iv) spraying the resulting solution onto a solid carrier having a temperature below the melting point of the vehicle,
v) mechanically working the resulting composition to obtain particles, i.e. a particulate material, and
vi) optionally subjecting the particulate material to conventional methods for preparing solid dosage forms.

In an important embodiment of the invention, at least part of the fibrate is present in the composition in the form of a solid dispersion including a molecular dispersion and a solid solution. Normally, about 10% or more such as, e.g., about 20% or more, about 30% or more, about 40% or more, about 50% or more, about 60% or more, about 70% or more, about 80% or more, about 90% or more such as, e.g., about 95% or more or about 100% w/w of the fibrate is present in the vehicle in the form of a solid dispersion, provided that at least about 80% w/w of the total amount of active substances is dissolved in the vehicle.

The first (fibrate-containing) composition may also be prepared using a dispersion of micronized fenofibrate, i.e. crystalline fenofibrate subjected to micronizing, for example in a conventional jet mill, in order to obtain a reduced crystalline particle size in the micron-range. Fenofibrate particles in the nano-range are also useful in the present invention.

A solid dispersion may be obtained in different ways e.g., by employing organic solvents or by dispersing or dissolving the active substance in another suitable medium (e.g. an oily material that is in liquid form at room temperature or at elevated temperatures). Solid dispersions (solvent method) are prepared by dissolving a physical mixture of the active substance (e.g. a drug substance) and the carrier in a common organic solvent, followed by evaporation of the solvent. The carrier is often a hydrophilic polymer. Suitable organic solvents include pharmaceutical acceptable solvent in which the active substance is soluble such as methanol, ethanol, methylene chloride, chloroform, ethylacetate, acetone or mixtures thereof.

The second solid (statin-containing) composition may be prepared by conventional wet granulation techniques as disclosed in the Examples below.

Suitable water-soluble carriers include polymers such as polyethylene glycol, poloxamers, polyoxyethylene stearates, poly-epsilon-caprolactone, polyvinylpyrrolidone (PVP), polyvinylpyrrolidone-polyvinylacetate copolymer PVP-PVA (Kollidon VA64), polymethacrylic polymers (Eudragit RS, Eudragit RL, Eudragit NE, Eudragit E) and polyvinyl alcohol (PVA), hydroxypropyl cellulose (HPC), hydroxypropyl methyl cellulose (HPMC), methyl cellulose, and poly(ethylene oxide) (PEO).

Polymers containing acidic functional groups may be suitable for solid dispersions, which release the active substance in a preferred pH range providing acceptable absorption in the intestines. Such polymers may be one or more selected from the group comprising hydroxypropyl methylcellulose phtalate (HMPCP), polyvinyl acetate phthalate (PVAP), hydroxypropylmethylcellulose acetate succinate (HPMCAS), alginate, carbomer, carboxymethylcellulose, methacrylic acid copolymer (Eudragit L, Eudragit S), shellac, cellulose acetate phthalate (CAP), starch glycolate, polacrylin, methyl cellulose acetate phthalate, hydroxypropylcellulose acetate phthalate, cellulose acetate terephthalate, cellulose acetate isophthalate and cellulose acetate trimellitate.

The weight ratio of active substance to polymer may be in a range of from about 3:1 to about 1:20. However, narrower ranges of from about 3:1 to about 1:5, such as, e.g., from about 1:1 to about 1:3 or about may also be used.

Apart from using the organic solvent based method, solid dispersion or solid solutions of one or more fibrates may be also obtained by dispersing and/or dissolving the active compound in the carrier composition used in the controlled agglomeration method. Stabilizing agents etc. may be added in order to ensure the stability of the solid dispersion/solution.

Fenofibrate and a statin may be combined in the composition or solid dosage form of the invention by using the following method: A fenofibrate granulate is prepared as disclosed in WO 2005/034920 and example 1 herein. A statin granulate is prepared using a conventional wet granulation method. The two granulates are mixed and either compressed into tablets or filled into hard gelatine capsules or sachets. The statin granulate may be entero-coated or coated with a protective coating, for example a film-forming polymer and stabilizers (antioxidants). The tablets might be sub-coated with a film-forming polymer before coating with the statin suspension below.

Examples of film polymers include water soluble agents such as hydroxypropylmethylcellulose, Metolose® (HPMC), hydroxypropylmethylcellulose, Klucel® (HPC), polyvinyl alcohol (PVA), polyvinylpyrrolidone (PVP) or combinations of PVA and PVP (Kollicoat® IR) and acid soluble acrylic polymer (Eudragit E, soluble in gastric juice).

Examples of antioxidants includes butylhydroxyanisol (BHA), ascorbyl palmitate, ascorbic acid or combinations of BHA, ascorbyl palmitate and citric acid. Wetting and pH adjusting agent might be included in the coating suspension Coating of the statin composition is performed in conventional coating equipment such as drum coater, perforated vessel or fluidized bed (Wurster insert).

### Solid Dosage Forms

The pharmaceutical composition of the invention is prepared in a solid dosage form which may be a single unit dosage form or in the form of a polydepot dosage form containing a multiplicity of individual units such as pellets, beads and/or granules.

Usually, the pharmaceutical composition in a solid dosage form of the invention is intended for administration via the oral, buccal or sublingual administration route.

The invention also relates to the above-mentioned presentation form. Within the scope of the invention are compositions/solid dosage forms that are intended to release the active substance in a fast release, a delayed release or modified release manner.

A useful solid dosage form comprises a pharmaceutical composition in particulate form as described above. The details and particulars disclosed under this main aspect of the invention apply mutatis mutandis to the other aspects of the invention. Accordingly, the properties with respect to increase in bioavailability, therapeutic and/or pharmacological response, changes in bioavailability parameters, reduction in adverse food effect as well as release of one or more fibrates etc. described and/or claimed herein for pharmaceutical compositions in particulate form are analogues for a solid dosage form according to the present invention.

The solid dosage form, i.e. in unit dosage form, comprises from about 100 to about 170 mg of fenofibrate, for example 100 mg or 110 mg or 120 mg or 130 mg or 145 mg or 160 mg of fenofibrate, and from about 5 to about 80 mg of statin or a pharmaceutically acceptable salt thereof, for example 5 mg or 10 mg or 20 mg or 40 mg or 80 mg of simvastatin or of atorvastatin.

In a preferred embodiment of the invention there is provided a pharmaceutical composition, in a single solid dosage form, comprising a fixed dose combination selected from the group consisting of atorvastatin 5 mg and fenofibrate 100 mg; atorvastatin 10 mg and fenofibrate 100 mg; atorvastatin 20 mg and fenofibrate 100 mg; atorvastatin 40 mg and fenofibrate 100 mg; atorvastatin 80 mg and fenofibrate 100 mg; atorvastatin 5 mg and fenofibrate 110 mg; atorvastatin 10 mg and fenofibrate 110 mg; atorvastatin 20 mg and fenofibrate 110 mg; atorvastatin 40 mg and fenofibrate 110 mg; atorvastatin 80 mg and fenofibrate 110 mg; atorvastatin 5 mg and fenofibrate 120 mg; atorvastatin 10 mg and fenofibrate 120 mg: atorvastatin 20 mg and fenofibrate 120 mg; atorvastatin 40 mg and fenofibrate 120 mg; and atorvastatin 80 mg and fenofibrate 120 mg; atorvastatin 5 mg and fenofibrate 130 mg; atorvastatin 10 mg and fenofibrate 130 mg: atorvastatin 20 mg and fenofibrate 130 mg; atorvastatin 40 mg and fenofibrate 130 mg; and atorvastatin 80 mg and fenofibrate 130 mg; atorvastatin 5 mg and fenofibrate 145 mg; atorvastatin 10 mg and fenofibrate 145 mg: atorvastatin 20 mg and fenofibrate 145 mg; atorvastatin 40 mg and fenofibrate 145 mg; and atorvastatin 80 mg and fenofibrate 145 mg.

In another preferred embodiment there is provided a pharmaceutical composition comprising, in a single solid dosage form, a fixed dose combination selected from the group consisting of simvastatin 5 mg and fenofibrate 100 mg; simvastatin 10 mg and fenofibrate 100 mg; simvastatin 20 mg and fenofibrate 100 mg; simvastatin 40 mg and fenofibrate 100 mg; simvastatin 80 mg and fenofibrate 100 mg; simvastatin 5 mg and fenofibrate 110 mg; simvastatin 10 mg and fenofibrate 110 mg; simvastatin 20 mg and fenofibrate 110 mg; simvastatin 40 mg and fenofibrate 110 mg; simvastatin 80 mg and fenofibrate 110 mg; simvastatin 5 mg and fenofibrate 120 mg; simvastatin 10 mg and fenofibrate 120 mg: simvastatin 20 mg and fenofibrate 120 mg; simvastatin 40 mg and fenofibrate 120 mg; and simvastatin 80 mg and fenofibrate 120 mg; simvastatin 5 mg and fenofibrate 130 mg; simvastatin 10 mg and fenofibrate 130 mg: simvastatin 20 mg and fenofibrate 130 mg; simvastatin 40 mg and fenofibrate 130 mg; simvastatin 80 mg and fenofibrate 130 mg; simvastatin 5 mg and fenofibrate 145 mg; simvastatin 10 mg and fenofibrate 145 mg: simvastatin 20 mg and fenofibrate 145 mg; simvastatin 40 mg and fenofibrate 145 mg; and simvastatin 80 mg and fenofibrate 145 mg.

The solid dosage forms comprising the pharmaceutical composition of the invention are very stable. For example, the fibrate is present in an amount of at least about 90%, or at least about 95%, or at least about 100%, relative to the amount prior to storage, when assayed after 3 months of storage at a temperature of about 40° C. and a relative humidity of about 75%. Also, the physical stability is very high as can be seen from the Examples below.

The solid dosage form according to the invention is obtained by processing the particulate material according to the invention by means of techniques well-known to a person skilled in the art. Usually, this involves further addition of one or more of the pharmaceutically acceptable excipients mentioned herein.

The composition or solid dosage form according to the invention may be designed to release fenofibrate and/or simvastatin/atorvastatin in any suitable manner provided that the increase in bioavailability is maintained. Thus, the active substance(s) may be released relatively fast in order to obtain an enhanced on-set of action, it may be released so as to follow zero or first order kinetics or it may be released in a controlled or modified manner in order to obtain a predetermined pattern of release. Plain formulations are also within the scope of the present invention.

The composition or solid dosage form according to the invention may also be coated with a film coating, an enteric coating, a modified release coating, a protective coating, an anti-adhesive coating etc.

A solid dosage form according to the invention may also be coated in order to obtain suitable properties e.g. with respect to release of the active substance. The coating may be applied on single unit dosage forms (e.g. tablets, capsules) or it may be applied on a polydepot dosage form or on its individual units.

Suitable coating materials are e.g. methylcellulose, hydroxypropylmethylcellulose, hydroxypropylcellulose, acrylic polymers, ethylcellulose, cellulose acetate phthalate, polyvinyl acetate phthalate, hydroxypropyl methylcellulose phthalate, polyvinylalcohol, sodium carboxymethylcellulose, cellulose acetate, cellulose acetate phthalate, gelatin, methacrylic acid copolymer, polyethylene glycol, shellac, sucrose, titanium dioxide, carnauba wax, microcrystalline wax, zein.

Plasticizers and other ingredients may be added in the coating material. The same or different active substance may also be added in the coating material. The pharmaceutical composition or a solid dosage form according to the invention is designed to release the fibrate in a suitable manner.

### Other Aspects of the Invention

A pharmaceutical composition or a solid dosage form according to the invention is designed to release the fibrate in a suitable manner. Specific release patterns as well as specific absorption patterns are mentioned below.

In specific embodiments, the fibrate and/or the statin is released from the composition within about 2 hours such as, e.g., within about 1.5 hours or within about 1 hour after oral administration, and/or about 50% w/w or more of the fibrate and/or the statin is released from the composition within about 30 min after oral administration, and/or about 50% w/w or more of the fibrate and/or the statin is released from the composition within about 20 min after oral administration, and/or about 60% w/w or more of the fibrate is released from the composition within about 1.5 hours after oral administration, and/or about 60% w/w or more of the fibrate and/or the statin is released from the composition within about 1 hour after oral administration, and/or about 70% w/w or more of the fibrate and/or the statin is released from the composition within about 1.5 hours after oral administration, and/or about 70% W/W or more of the fibrate and/or the statin is released from the composition within about 1 hour after oral administration, and/or about 85% w/w or more of the fibrate and/or the statin is released from the composition within about 45 min when tested in an in vitro dissolution test according to USP dissolution test (paddle) employing water as dissolution medium, 100 rpm and a temperature of about 37° C.

In another embodiment about 50% w/w or more of the fibrate and/or the statin is released from the composition within about 20 min, 15 min or 10 min, and/or about 60% w/w or more of the fibrate and/or the statin is released from the composition within about 20 min or 15 min, and/or about 70% w/w or more of the fibrate and/or the statin is released from the composition within about 20 min or 15 min, when tested in an in vitro dissolution test according to USP dissolution test (paddle) employing water as dissolution medium, 100 rpm and a temperature of about 37° C.

In a still further embodiment about 50% w/w or more of the fibrate and/or the statin contained in the composition is absorbed within about 8 hours, 7 hours, 6 hours or 5 hours, and/or about 60% w/w or more of the fibrate and/or statin contained in the composition is absorbed within about 8 hours or 7 hours after oral administration, and/or about 60% w/w or more of the fibrate contained in the composition is absorbed within about 7 hours after oral administration, and/or about 70% w/w or more of the fibrate contained in the composition is absorbed within about 8 hours or 7 hours after oral administration.

The details and particulars disclosed under this main aspect of the invention apply mutatis mutandis to the other aspects of the invention. Accordingly, the properties with respect to increase in bioavailability, changes in bioavailability parameters, reduction in adverse food effect as well as release of one or more fibrates etc. described and/or claimed herein for pharmaceutical compositions in particulate form are analogues for a solid dosage form according to the present invention.

### Materials and Methods

### Materials

### Fenofibrate (supplied by Sigma)

### Atorvastatin magnesium, atorvastatin calcium (supplied by Biocon)

### Simvastatin (supplied Biocon)

### Lactose monohydrate 200 mesh (from DMV)

Mannitol. Pearlitol 50 C (from Roquette, France)

### Polyethylene glycol 6000, Pluracol® E6000 (from BASF)

### Poloxamer 188, Pluronic® F-68 (from BASF)

### Avicel PH200 (microcrystalline cellulose) (from FMC)

### Ac-di-sol (croscarmellose sodium, from FMC Corp., U.S.A.)

### Trometamol (from Dow France (Angus))

### Klucel (from Hercules Inc, U.S.A.)

### Magnesium stearate

Tablets, capsules or granules may be enteric coated with different types of polymers such as hydroxypropylmethylcellulose acetate succinate (Aqoat), cellulose acetate phthalate CAP, hydroxypropylmethylcellulose phtalate HPMCP or methacrylic acid copolymers such as Eudragit L30D, Eudragit 100/S, Eudragit 100/L.

### Equipment

Laboratory scale fluid bed equipment: Strea-1.
The melt feed unit is a prototype composed of separate units for heating of air supplies for the atomizer, pressure tank and feeding tube. Granulate was sieved manually and mixed with extragranular excipients in a Turbula mixer.

### Tablet compression was performed on a multilayer (bi-layer) tablet machine.

### Methods

The fenofibrate drug may be dissolved into the melted vehicle(s) and applied on the particulate carrier(s) as follows:

The vehicle(s) was melted in a beaker placed in a microwave oven. The beaker was transferred to a temperature controlled heating plate supplied with magnetic stirring. Fenofibrate was dissolved slowly in the melt at a temperature of 75° C. under magnetic stirring. The hot solution was transferred to the pressure tank for melt spray application onto the carrier in the fluid bed. The granulate product was discharged from the fluid bed and sieved through sieve 0.7 mm or 1.0 mm manually. The sieved product was blended with magnesium stearate for 0.5 min in a Turbula mixer. If an extragranular phase has to be incorporated, the extragranular phase was premixed with the granulate in 3 minutes in a Turbula mixer.

### Release Test

A fat-soluble colorant Sudan II (BDH Gur®) obtained from BDH VWR International 14.3 mg was dissolved in 50.0 g viscoleo (fractionated medium chain triglycerides).

10 g of the oil was added to 10.0 g of the solid pharmaceutically acceptable material to be tested for use according to the present invention and mixed until the oil was fully absorbed in the solid material. The mixture was subsequently sieved through sieve 0.3 mm to achieve a homogeneous mixture.

1.00 g of the mixture was transferred to a centrifugal tube and 3.00 ml of water was added. The suspension was mixed in a blood sample turner for 1 hour and subsequently centrifuged for 10 minutes at 5000 rpm. The upper phase of oil and water was transferred carefully to a beaker and the water was evaporated in an oven at 80° C. until constant weight. The amount of oil released from the solid material was calculated on basis of the weight of the remaining after evaporation of the water phase.

### Disintegration Test

The disintegration time was determined according to the method described in to Ph. Eur.

### Dissolution Test

The test was performed in accordance with Ph. Eur 2.9.3 using the paddle apparatus.

The quantification was performed using HPLC with UV-detection.

| | |
|---|---|
| Medium: | 900 ml water with 0.75% sodium lauryl sulfate |
| | (SLS) |
| Rotation speed: | 50 rpm |
| Temperature: | 37° C. |
| Sampling time: | 10, 20, 30, 45 and 60 minutes |
| Acceptance criteria: | >75% at 45 minutes (for the stability study) |

### Test for Impurities

### Sample Preparation for Simvastatin:

10 tablets were grounded and about 957 mg of grounded tablet material was placed in a 25 mL volumetric flask. 5 mL of water was added and the mixture was ultrasonicated for 10 minutes. Acetonitrile was added up to a total volumen of 25 mL and the mixture was ultrasonicated for further 10 minutes, followed by filtration (0.45 micrometer filter). The resulting material was diluted ×25 for quantification.

### Sample Preparation for Atorvastatin:

10 tablets were grounded and about 963 mg±10 mg of grounded tablet material was placed in a 25 mL volumetric flask. 5 mL of water and 15 ml acetonitrile was added and the mixture was stirred on a magnetic stirrer for 60 minutes. Acetonitrile was added up to a total volume of 25 mL and filtrated through 0.45 μm filter. The sample is diluted ×25 for quantification. Both concentrated and diluted sample was injected into the HPLC system HPLC:

The sample was subjected to HPLC analysis on a Shimadzu 2010A with auto sampler cooling and dual wavelength UV detector.

### Eluent A: 10.6 mM formic acid (in water). Eluent B: 10.6 mM formic acid (in acetonitrile).

### Column: varian Pursuit C18 3 micro, 150×3.0 mm

Oven temperature: 30° C.
Injection volume: 15 microliter

### Flow: 0.5 mL/min

### Gradient:

| Time (min) | Eluent A | Eluent B |
|---|---|---|
| 0.0 | 60 | 40 |
| 0.5 | 60 | 40 |
| 30.0 | 15 | 85 |
| 35.0 | 60 | 40 |
| 40.0 | 60 | 40 |

### Detection wavelength—fenofibrate: 295 nm

### Detection wavelength—simvastatin/atorvastatin: 240 nm

### Determination of Weight Variation

The tablets prepared in the Examples herein were subject to a test for weight variation performed in accordance with Ph. Eur.

### Determination of Average Tablet Hardness

The tablets prepared in the Examples herein were subject to at test for tablet hardness employing Schleuniger Model 6D apparatus and performed in accordance with the general instructions for the apparatus.

### Determination of Solid Solution

According to the present invention, the fibrate is dissolved in a vehicle. In order to substantiate this, a test involving differential scanning calometry is performed. The test is performed on the particulate composition, solid dosage form or mixture of vehicle and fibrate (after the solid solution is supposed to form). Standard DSC equipment connected to a PC is used.

Sample size: 10 mg in alu pans
Heating rate: 5° C./min from 27° C. to 110° C.

### Evaluation: The fibrate is considered to be in dissolved state or non-crystalline if no fibrate endotherm peak is observed and if the melting interval does not significantly shift compared with the vehicle alone.

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

### EXAMPLE 1

### Preparation of Fenofibrate Granulate

The following fenofibrate granulate denoted 1A was prepared as described above under Methods and in WO-A-2005/034920, which is incorporated by reference in its entirety. ‘mg/tablet’ denotes the amounts present in the pharmaceutical composition of the invention in a single solid dosage form (a tablet):

1A

| Substance | Ingredients | % | mg/tablet |
|---|---|---|---|
| Drug | Fenofibrate | 19.6 | 160.00 |
| Carrier | Lactose | 43.6 | 356.50 |
| Vehicle | PEG 6000 | 25.4 | 208.20 |
| Vehicle | Poloxamer 188 | 10.9 | 89.20 |
| Excipient | Magnesium stearate | 0.5 | 4.10 |
| | | 100.0 | 818.00 |

The following fenofibrate granulates denoted 1B, 1C, 1D and 1E were prepared as described above under Methods and in WO-A-2005/034920, which is incorporated by reference in its entirety. ‘mg/tablet’ denotes the amounts present in the pharmaceutical composition of the invention in a single solid dosage form (a tablet):

| Sub- | | 1B | 1C | 1D | 1E | 1F | 1G | 1H |
|---|---|---|---|---|---|---|---|---|
| stance | Ingredient | mg | mg | mg | mg | mg | mg | mg |
| Drug | Fenofibrate | 130 | 43 | 48 | 145 | 120 | 110 | 100 |
| Vehicle | PEG6000 | 169 | 56 | 62 | 189 | 157 | 144 | 131 |
| 1 | | | | | | | | |
| Vehicle | Poloxamer | 72 | 24 | 27 | 81 | 67 | 61 | 56 |
| 2 | 188 | | | | | | | |
| Carrier | Lactose | 304 | 101 | 112 | 339 | 282 | 258 | 235 |
| Excip- | Mg | 1.3 | 0.5 | 2.5 | 7.6 | 6.3 | 5.8 | 5.3 |
| ients | stearate | | | | | | | |

### EXAMPLE 2

### Preparation of Simvastatin Granulate

The following simvastatin granulate denoted 2A was prepared using a conventional wet granulation method.

‘mg/tablet’ denotes the amounts present in the pharmaceutical composition of the invention in a single solid dosage form (a tablet):

| Substance | Ingredient | % | mg/tablet |
|---|---|---|---|
| Drug | Simvastatin | 4.9 | 10.0 |
| Carrier | Lactose 350 mesh | 33.0 | 68.0 |
| Excipients | Magnesium stearate | 0.5 | 1.0 |
| | Talc | 0.2 | 0.4 |
| | Starch 1500 | 9.8 | 20.0 |
| | Klucel (hydroxy propyl cellulose) | 1.5 | 3.0 |
| | Citric acid/BHA (antioxidant) | 1.1 | 2.5 |
| | Avicel PH200 (microcryst. cellulose) | 49.5 | 102.0 |

### EXAMPLE 3

### Preparation of Atorvastatin Granulate

The following atorvastatin granulate denoted 3A was prepared in a conventional manner using wet granulation, i.e. mixing atorvastatin, lactose (carrier) and calcium carbonate (stabilizer), adding the appropriate amount of hydroxypropyl cellulose (Klucel; binder) and natrium carboxymethyl cellulose (Ac-di-sol; disintegrant), adding sterile water to the mixture, mixing and drying off the water, sifting the dried mixture and adding magnesium stearate (lubricant) and microcrystalline cellulose (Avicel).

The following atorvastatin granulate denoted 3B was prepared in a conventional manner using wet granulation: A binder solution is prepared by dissolving hydroxypropyl cellulose (binder) and trometamol (stabilizer) in water (surfactant may be added, e.g. Polysorbat 80). Atorvastatin, mannitol (carrier), hydroxypropyl cellulose (binder) and microcrystalline cellulose (Avicel; filler) is transferred to a high shear mixer. The dry ingredients are premixed for 2 minutes, followed by addition of the binder solution at 150 rpm (impeller) and 2000 rpm (chopper) to form a wet mass. Water is added and the mixture is mixed for 2 minutes, resulting in granule formation. The wet granulate is sieved (1.0 mm round opening) and dried in a fluid bed. The dry granulate is sieved (1.0 mm round opening). ‘%’ denotes percentage of granulate.

‘mg/tablet’ denotes the amounts present in the pharmaceutical composition of the invention in a single solid dosage form (a tablet):

| | | 3A | | 3B | |
|---|---|---|---|---|---|
| Substance | Ingredient | % | mg/tablet | % | mg/tablet |
| Drug | Atorvastatin | 5.3 | 10.9 | 14.6 | 44 |
| | magnesium | | | | |
| Carrier | Lactose 200 mesh | 16.1 | 32.8 | — | — |
| | Mannitol | — | — | 41 | 122 |
| | (Pearlitol 50C) | | | | |
| Excipients | Magnesium stearate | 0.5 | 1.00 | 0.5 | 1.5 |
| | Ac-di-sol | 5.0 | 10.2 | — | — |
| | Calcium carbonate | 16.2 | 33.0 | — | — |
| | Klucel | 1.5 | 3.0 | 2.4 | 7 |
| | Polysorbate 80 | 0.4 | 0.6 | 0.8 | 2.4 |
| | Avicel | 55.0 | 111.7 | 40 | 119 |
| | Trometamol | — | — | 0.8 | 2.5 |

### EXAMPLE 4

### Tablet of the Invention—Atorvastatin

A two-layer tablet denoted 4E was prepared in a conventional manner in a tableting machine (manufactured by Fette GmbH, Germany) using fenofibrate granulate 1E of example 1 and atorvastatin granulate 3B of example 3, the resulting tablet having a weight of about 1060 mg.

A two-layer tablet denoted 4B was prepared in a conventional manner in a tableting machine (manufactured by Fette GmbH, Germany) using fenofibrate granulate 1F of example 1 and atorvastatin granulate 3B of example 3, the resulting tablet having a weight of about 930 mg.

### EXAMPLE 5

### Tablet of the Invention—Simvastatin

A two-layer tablet denoted 5E was prepared in a conventional manner in a tableting machine (manufactured by Fette GmbH, Germany) using fenofibrate granulate 1E of example 1 and simvastatin granulate 2A of example 2, the resulting tablet having a weight of about 938 mg.

A two-layer tablet denoted 5B was prepared in a conventional manner in a tableting machine (manufactured by Fette GmbH, Germany) using fenofibrate granulate 1B of example 1 and simvastatin granulate 2A of example 2, the resulting tablet having a weight of about 853 mg.

### EXAMPLE 6

### Comparison Example—Stability of Pharmaceutical Composition of the Invention (Simvastatin)

It is known that simvastatin may degrade to the corresponding hydroxy acid upon storage, thus creating ‘impurities’ in the pharmaceutical composition comprising simvastatin.

Stability of the fixed dose fenofibrate and simvastatin tablets prepared according to the invention (example 5) was measured as described above (test for impurities) after 1 month storage at 25° C. and 60% RH. The comparison tablet was a tablet prepared by mixing the fenofibrate granulate 1E (example 1) and the simvastatin granulate 2A (example 2) and compressing the combined granulate into a tablet.

Results:

| | Hydroxy acid | |
|---|---|---|
| Formulation | % of simvastatin | Index |
| Comparison (granulate mix) | 3.9 | 1857 |
| Tablet with pharmaceutical | 0.2 | 100 |
| composition of the invention | | |

The result clearly demonstrates that a single solid dosage form prepared according to the invention (a two-layer tablet in this embodiment) is significantly more stable than a tablet prepared from a simple mix of similar active substance granulates.

### EXAMPLE 7

### Stability of Pharmaceutical Composition of the Invention (Atorvastatin)

Stability of the fixed dose fenofibrate and atorvastatin tablets prepared according to the invention (example 4) was measured as described above (test for presence of atorvastatin in lactone form, varying amounts of trometamol stabilizer) after 1 month storage at 40° C. and 75% RH.

Results:

| | Trometanole added | | |
|---|---|---|---|
| | 1% w/w | 2% w/w | 5% w/w |
| Lactone content | <0.05% | <0.05% | <0.05% |

1. A composition for oral administration comprising a fixed dose combination of a first solid composition containing fenofibrate as the active substance and second solid composition containing an HMG-CoA reductase inhibitor as the active substance, wherein the first and the second composition are present in separate entities in a single solid dosage form.

2. The composition according to claim 1, wherein the first solid composition is in the form of granulate, granules, grains, beads or pellets.

3. The composition according to claim 1, wherein the second solid composition is in the form of granulate, granules, grains, beads or pellets.

4. The composition according to claim 3, wherein the granules, granulate, grains, beads or pellets are entero-coated.

5. The composition according to claim 3, wherein the granules, granulate, grains, beads or pellets are coated with a protective coating.

6. The composition according to claim 1 in the form of a capsule or a sachet.

7. The composition according to claim 1 in the form of a tablet.

8. The composition according to claim 7, wherein the first and second compositions are present in the tablet in separate layers.

9. The composition according to claim 8, wherein a layer comprising the first composition is separated from a layer comprising the second composition by an intermediate, inactive layer.

10. The composition according to claim 1, wherein the HMG-CoA reductase inhibitor is a statin selected from the group consisting of atorvastatin, lovastatin, pravastatin, simvastatin, rosuvastatin, fluvastatin and pitavastatin.

11. The composition according to claim 10, wherein the HMG-CoA reductase inhibitor is simvastatin.

12. The composition according to claim 11 comprising a fixed dose combination selected from the group consisting of simvastatin 5 mg and fenofibrate 100 mg; simvastatin 10 mg and fenofibrate 100 mg; simvastatin 20 mg and fenofibrate 100 mg; simvastatin 40 mg and fenofibrate 100 mg; simvastatin 80 mg and fenofibrate 100 mg; simvastatin 5 mg and fenofibrate 110 mg; simvastatin 10 mg and fenofibrate 110 mg; simvastatin 20 mg and fenofibrate 110 mg; simvastatin 40 mg and fenofibrate 110 mg; simvastatin 80 mg and fenofibrate 110 mg; simvastatin 5 mg and fenofibrate 120 mg; simvastatin 10 mg and fenofibrate 120 mg: simvastatin 20 mg and fenofibrate 120 mg; simvastatin 40 mg and fenofibrate 120 mg; and simvastatin 80 mg and fenofibrate 120 mg; simvastatin 5 mg and fenofibrate 130 mg; simvastatin 10 mg and fenofibrate 130 mg simvastatin 20 mg and fenofibrate 130 mg; simvastatin 40 mg and fenofibrate 130 mg; simvastatin 80 mg and fenofibrate 130 mg; simvastatin 5 mg and fenofibrate 145 mg; simvastatin 10 mg and fenofibrate 145 mg: simvastatin 20 mg and fenofibrate 145 mg; simvastatin 40 mg and fenofibrate 145 mg; and simvastatin 80 mg and fenofibrate 145 mg.

13. The composition according to claim 10, wherein the HMG-CoA reductase inhibitor is atorvastatin.

14. The composition according to claim 13, wherein the atorvastatin is selected from the group consisting of crystalline atorvastatin calcium, amorphous atorvastatin calcium, crystalline atorvastatin magnesium, amorphous atorvastatin magnesium, a mixture of amorphous and crystalline atorvastatin calcium and a mixture of amorphous and crystalline atorvastatin magnesium.

15. The composition according to claim 13, wherein the atorvastatin is crystalline atorvastatin magnesium.

16. The composition according to claim 13 comprising a fixed dose combination selected from the group consisting of atorvastatin 5 mg and fenofibrate 100 mg; atorvastatin 10 mg and fenofibrate 100 mg; atorvastatin 20 mg and fenofibrate 100 mg; atorvastatin 40 mg and fenofibrate 100 mg; atorvastatin 80 mg and fenofibrate 100 mg; atorvastatin 5 mg and fenofibrate 110 mg; atorvastatin 10 mg and fenofibrate 110 mg; atorvastatin 20 mg and fenofibrate 110 mg; atorvastatin 40 mg and fenofibrate 110 mg; atorvastatin 80 mg and fenofibrate 110 mg; atorvastatin 5 mg and fenofibrate 120 mg; atorvastatin 10 mg and fenofibrate 120 mg: atorvastatin 20 mg and fenofibrate 120 mg; atorvastatin 40 mg and fenofibrate 120 mg; and atorvastatin 80 mg and fenofibrate 120 mg; atorvastatin 5 mg and fenofibrate 130 mg; atorvastatin 10 mg and fenofibrate 130 mg: atorvastatin 20 mg and fenofibrate 130 mg; atorvastatin 40 mg and fenofibrate 130 mg; and atorvastatin 80 mg and fenofibrate 130 mg; atorvastatin 5 mg and fenofibrate 145 mg; atorvastatin 10 mg and fenofibrate 145 mg: atorvastatin 20 mg and fenofibrate 145 mg; atorvastatin 40 mg and fenofibrate 145 mg; and atorvastatin 80 mg and fenofibrate 145 mg.

17. The composition according to claim 13 which further comprises a stabilizer capable of providing a microenvironment for atorvastatin having a pH of at least about 5.

18. The composition according to claim 13 which further comprises a stabilizer capable of providing a microenvironment for atorvastatin having a pH of at least about 6.

19. The composition according to claim 13 which further comprises a stabilizer selected from the group consisting of inorganic alkalizing compounds.

20. The composition according to claim 19, wherein the stabilizer is selected from the group consisting of metal salts, alkaline earth metal salts, talc and bentonite.

21. The composition according to claim 19, wherein the stabilizer is selected from the group consisting of calcium salts (calcium carbonate, calcium hydroxide, di calcium phosphate, tri calcium phosphate), magnesium salts (magnesium carbonate, magnesium hydroxide, magnesium silicate, magnesium aluminate, aluminum magnesium hydroxide), lithium salts (lithium hydroxide), potassium salts (potassium hydroxide) and sodium salts (sodium bicarbonate, sodium borate, sodium carbonate, sodium hydroxide).

22. The composition according to claim 13 which further comprises a stabilizer selected from the group consisting of organic alkalizing compounds.

23. The composition according to claim 22, wherein the stabilizer is selected from the group consisting of amines, amides and ammonium compounds.

24. The composition according to claim 22, wherein the stabilizer is selected from the group consisting of ammonia, ammonium lactate, ammonium bicarbonate, ammonium hydroxide, ammonium phosphate dibasic, mono ethanolamine, di ethanolamine, tri ethanolamine, tri hydroxymethylaminomethane, ethylenediamine, N-methyl glucamide, 6N-methyl glucamine, meglucamine, L-lysine and 2-amino-2-(hydroxymethyl)-1,3-propanediol.

25. The composition according to claim 17, wherein the stabilizer is 2-amino-2-(hydroxymethyl)-1,3-propanediol.

26. The composition according to claim 1, wherein the second composition comprises atorvastatin and from about 0.01% w/w to about 5% w/w of 2-amino-2-(hydroxymethyl)-1,3-propanediol.

27. The composition according to claim 1, wherein the first or the second composition further comprises acceptable excipients.

28. The composition according to claim 1, wherein the first composition comprises micronized crystalline fenofibrate.

29. The composition according to claim 1, wherein the first composition comprises a solid solution of fenofibrate dissolved in a vehicle comprising polyethylene glycol (PEG).

30. The composition according to claim 27, wherein the first composition comprises a solid solution of fenofibrate dissolved in a vehicle comprising polyethylene glycol 6000 (PEG 6000) and poloxamer 188.

31. The composition according to claim 27, wherein the first composition comprises lactose as a carrier.

32. The composition according to claim 27, wherein the first composition comprises magnesium stearate as a lubricant.

33. The composition according to claim 1, wherein the second composition comprises simvastatin and lactose as a carrier.

34. The composition according to claim 1, wherein the second composition comprises atorvastatin magnesium and mannitol as a carrier.

35. The composition according to claim 27, wherein the second composition comprises magnesium stearate as a lubricant.

36. The composition according to claim 27, wherein the second composition comprises starch as a disintegrant.

37. The composition according to claim 27, wherein the second composition comprises one or more antioxidants selected from the group consisting of ascorbic acid, citric acid and butyl hydroxyl anisole.

38. The composition according to claim 27, wherein the second composition comprises microcrystalline cellulose as a filler.

39. The composition according to claim 1, wherein the single solid dosage form is a two-layer tablet prepared by compressing the first composition in the form of granulate together with the second composition in the form of granulate

40. The composition according to claim 1, wherein the single solid dosage form is a two-layer tablet prepared by compressing the first composition in the form of granulate together with the second composition in the form of granulate having a protective coating.

41. The composition according to claim 1, wherein the single solid dosage form is a two-layer tablet prepared by compressing the first composition in the form of granulate together with the second composition in the form of entero-coated granulate.

42. The composition according to claim 1 containing not more than 0.5% atorvastatin in lactone form after storage at 40° C. and 75% relative humidity for 1 month.

43. The composition according to claim 1 containing not more than 0.1% atorvastatin in lactone form after storage at 40° C. and 75% relative humidity for 1 month.

44. The composition according to claim 1 containing not more than 0.05% atorvastatin in lactone form after storage at 40° C. and 75% relative humidity for 1 month.

45. The composition according to claim 1 for the treatment of a subject suffering from atherosclerosis, hyperlipidemia, and/or hypercholesterolemia.

46. The composition according to claim 43 for the treatment of a human subject.

47. A method for preparing a tablet comprising a first solid composition containing fenofibrate as the active substance and second solid composition containing an HMG-CoA reductase inhibitor as the active substance, the first and the second composition being present in separate entities, which method comprises the steps of:
i) preparing the first solid composition by dissolving fenofibrate in a vehicle and spraying the resulting liquid solution on a solid carrier in a controlled agglomeration process, optionally mixing the agglomerated particles with a lubricant, and mixing the agglomerated particles to form a granulate,
ii) preparing the second solid composition by wet granulation, and
iii) compressing the first and second compositions into a multilayer tablet, the first and second compositions being present in separate layers.

48. A single solid dosage form comprising a composition for oral administration comprising a fixed dose combination of a first solid composition containing fenofibrate as the active substance and second solid composition containing an HMG-CoA reductase inhibitor as the active substance, wherein the first and the second composition are present in separate entities.
